(19) United States
(12) Patent Application Publication
(10) Pub. No.: US 2007/0213677 A1
(43) Pub. Date: Sep. 13, 2007

(54) METHOD OF HYPERBARIC PERCUTANEOUS OXYGEN THERAPY BY SPRAYING FLUOROCARBONS

(75) Inventor: Jean Clement Edouard Ghislain Makal, Binche (BE)

Correspondence Address: SUGHRUE MION, PLLC, 2100 PENNSYLVANIA AVENUE, N.W., SUITE 800, WASHINGTON, DC, 20037, US

(73) Assignee: MC ANTON HOLDINGS S.A.

(21) Appl. No.: 11/573,818

(22) PCT Filed: Aug. 5, 2005
(86) PCT No.: PCT/IB05/02829

§ 371 Date: Feb. 16, 2007

(30) Foreign Application Priority Data
Aug. 18, 2004 (BE) 2004/0403

(51) Int. Cl. A61M 35/00

(52) U.S. Cl. 604/289; 604/310

(57) ABSTRACT

The invention relates to a method for transcutaneous medical, physiotherapeutical or cosmetic treatment of the skin and subjacent organs, comprising a step which consists in projecting a pressurized gas onto the area that is to be treated, with the aid of a projection nozzle. Use of said method promotes penetration of the skin by oxygen-carrying fluorocarbons, enabling oversaturation of said molecules with oxygen and the creation of a massage effect.

The invention relates to a method for the medical, physiotherapeutic or cosmetic treatment of the skin and subjacent organs of a human or animal subject via the percutaneous route, and also to a device for implementing such a method and to a handpiece for such a device. Various methods for rejuvenating the skin are already well known: abrasive peeling, chemical peeling, laser treatment, local skin injections and dermatological implants. These methods are painful and may give rise to side effects: intolerance, allergy, burns and scars.

In rheumatology, physiotherapeutic techniques such as massages, mud baths, heat and exercise aim to heat up the tissues, activate circulation and promote better oxygenation of the skin, muscles and joints.

However, the oxygenation of the tissues is limited by the duration of the physical effect achieved and by the pre-existing circulatory state.

In dermatology, many skin lesions could benefit from an additional supply of oxygen. At present, this is generally only possible by using vasodilatory medicaments, although the effect of these is limited or even contraindicated in the case of tissue inflammation.

In medicine, tissue oxygenation is a technique used in intensive care and essentially via the respiratory route.

Recently, the development of an artificial haemoglobin substitute has opened up the possibility of injectable oxygen therapy using fluorocarbons. Some of these molecules, such as perfluorodecalin for example, are capable of passing through the skin and supplying oxygen locally and remotely.

In 1989, IWAI T. et al demonstrated the beneficial effect of oxygenated baths enriched with perfluorocarbon for the treatment of ischemic ulcers of the lower limbs.

However, no technique to date has used hyperbaric percutaneous oxygen therapy using an oxygen carrier gas.

The invention proposes a method for the cosmetic, aesthetic, dermatological, physiotherapeutic, rheumatological and medical treatment of an area of human or animal skin, designed to improve tissue oxygenation and cell metabolism and thus to allow healing, cicatrisation and tissue rejuvenation.

Implementation of this method makes it possible to promote the penetration of small neutral oxygen carrier molecules through the skin, without resulting in any side effect. It also makes it possible to saturate these carrier molecules with oxygen. The treatment not only has no side effect but also it is pleasantly perceived as a gentle massage with no friction, the temperature and intensity of which can be adjusted. The subjects can therefore return to their activities immediately.

According to a first aspect, the invention proposes a method for the treatment, in particular the cosmetic treatment, of an area of skin, comprising the step consisting in spraying under pressure a cold, warm or hot gas which is enriched with oxygen and/or nitrogen monoxide or other medical gases onto the area that is to be treated, by means of a spray nozzle.

According to a second aspect, the invention relates to a device for percutaneous treatment, comprising a spray nozzle and comprising means for pressurising a gas and at least one conduit arranged between the pressurisation means and the spray nozzle.

According to a third aspect, the invention comprises a handpiece comprising a valve for opening and closing the pressurised gas circuit, a control knob for regulating the temperature of the gas, and a control knob for controlling an electrovalve which regulates the flow rate of the gas.

The subject matters and advantages of the invention will emerge from the following description, which is given with reference to the appended drawings, in which:

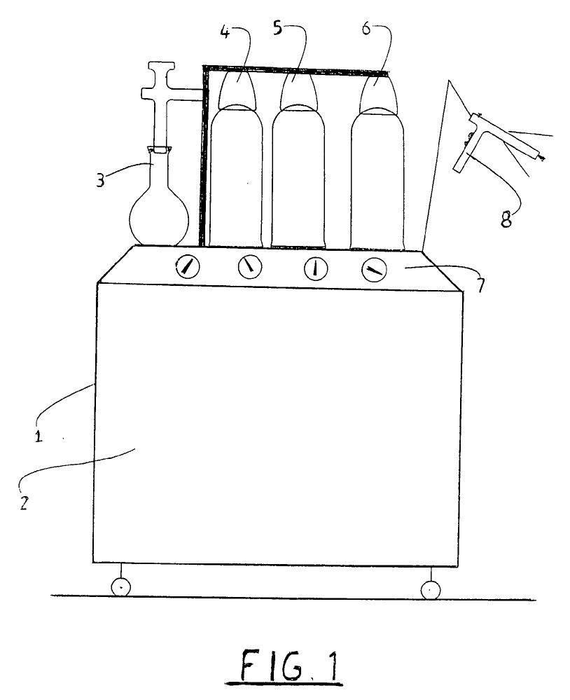

FIG. 1 shows a schematic front view of an implementation device according to the invention;

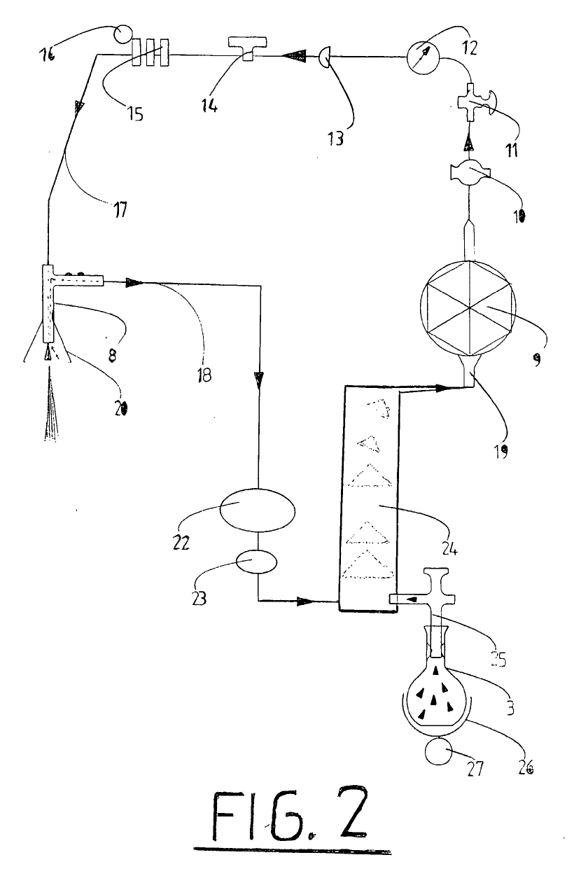

FIG. 2 is a schematic view of the elements which constitute the device of FIG. 1, according to an embodiment in which the device operates as a semi-closed circuit;

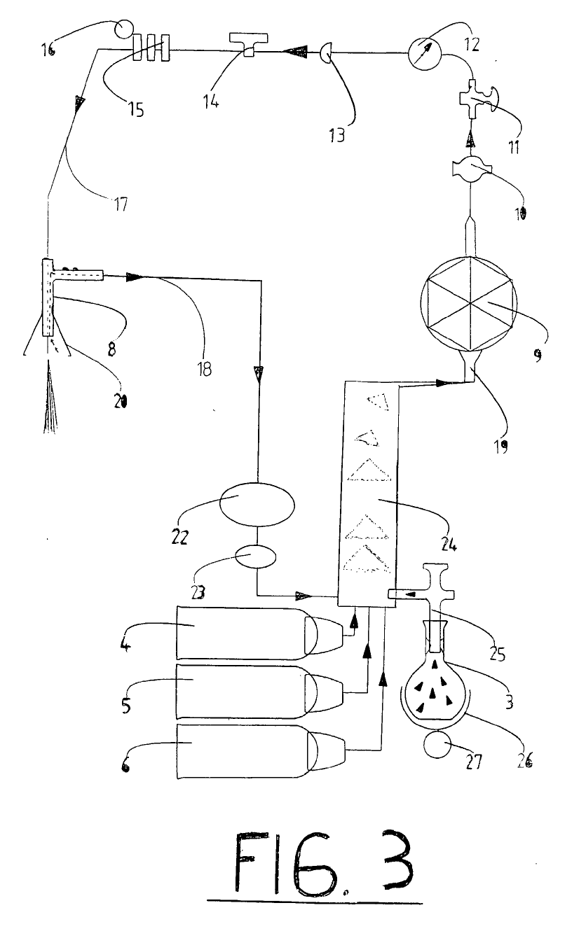

FIG. 3 is a schematic view of the elements which shows an embodiment in which the device comprises cylinders of various gases intended to be sprayed onto the skin;

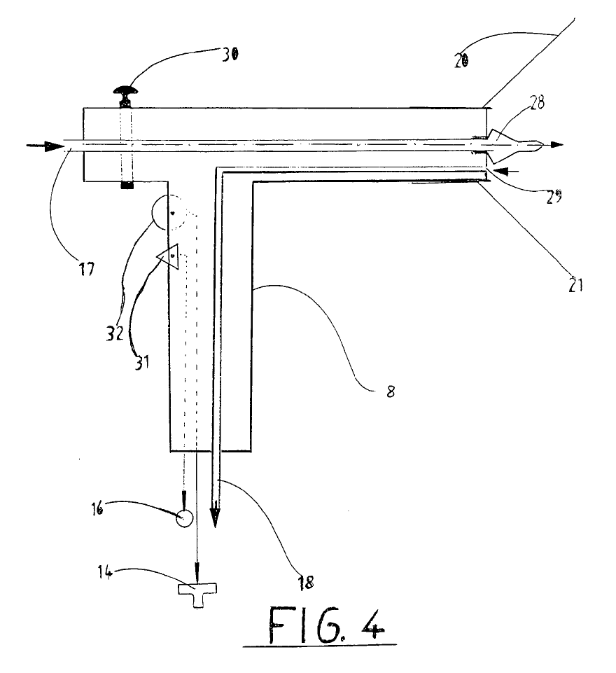

FIG. 4 is a sectional view of a handpiece which forms part of FIG. 1, according to an embodiment comprising a spray nozzle for spraying a pressurised gas.

The method according to the invention is designed to oversaturate perfluorocarbon molecules with oxygen and/or nitrogen monoxide or other medical gases and to promote the passage of said molecules through the skin by means of hyperbaric spraying.

According to the invention, this method comprises the step consisting in spraying a pressurised gas onto an area of skin that is to be treated, by means of a nozzle.

This method is carried out using a device (1) such as that shown in FIG. 1. This device comprises a housing (2) which contains means for pressurising a gas and means for aspirating said gas, a flask (3) which contains perfluorocarbons, a cylinder of oxygen (4), a cylinder of nitrogen monoxide (5), a cylinder of another medical gas (6), a control panel (7) and a handpiece (8) intended for the practitioner in order to carry out said method.

According to one embodiment, shown in FIG. 2, the means for pressurising the gas comprise a suction and compression pump (9) of the piston or membrane type, which is preferably able to pressurise the gas to a pressure ranging between 0.01 and 20 bar. Said pressurisation means are associated with means for controlling and measuring the pressure and temperature which comprise, in the flow direction of the gas, a damper (10), a pressure switch (11), a pressure gauge (12), a decompressor (13), an electrovalve (14) and a heating resistor (15) designed to heat the sprayed gas, and a thermostat (16).

A conduit (17) conveys the pressurised gas to the handpiece (8), via which said gas is sprayed onto an area of skin.

After having been sprayed onto the skin, the gas is partially aspirated through a conduit (18) connected to the inlet of the suction pump (19).

The handpiece (8) comprises a means (20) for recovering the gas, said means being in the shape of a cone, the base of which is oriented towards the skin and the apex of which is extended by an end piece (21) which can be screwed or pushed onto the end of the handpiece (8). Said means of recovering the gas is in communication with the conduit (18).

The conduit (18) also comprises filtration means for filtering macroscopic particles (22) and microscopic particles (23) so as to ensure that the aspirated gases are clean and sterile. It is on this conduit (18) that the means for mixing and saturating the sprayed gases are installed, said means being represented by a chamber (24).

Fluorocarbons have a vapour pressure which is respectively 12.7 mmHg for perfluorodecalin and 20 mmHg for perfluorotripropylamine at 37° C. The fluorocarbon vapour exits from a flask (3) and enters the chamber (24) via a connection means (25).

The flask (3) is heated by a bain-marie (26) which is designed to increase the vapour pressure of the fluorocarbons in the chamber (24). The bain-marie (26) has a means for controlling the temperature (27).

According to an embodiment shown in FIG. 3, pure oxygen from a cylinder (4) and/or nitrogen monoxide from a cylinder (5) or from a cylinder of another medical gas (6) are sprayed into the chamber (24) so as to saturate the fluorocarbon molecules with oxygen and/or nitrogen monoxide or another medical gas.

FIG. 4 shows the handpiece (8) which surrounds the conduit (17) connected to the nozzle (27), the aspirating cone (20) connected to the conduit (18). For this arrangement, the aspirating cone (20) is in communication with the conduit (18) via a channel (29).

This handpiece comprises the various control means comprising a valve (30) for opening or closing the pressurised gas circuit, an adjustment knob (31) for adjusting the thermostat (16) which controls the temperature of the sprayed gas in the system, and a control knob (32) for controlling the electrovalve (14) which regulates the flow rate of the sprayed gas.

One embodiment of the method according to the invention will now be described.

The skin of the subject to be treated is cleaned using liquid soap, following by a step of light peeling using glycolic acid in the form of a 70% gel buffered to pH 3. This step makes it possible for the fluorocarbon molecules to penetrate more easily into the skin. The fluorocarbon gas is prepared in a flask which is partially filled with a liquid fluorocarbon emulsion, commercially available under the trade name Fluosol DA for example.

The flask of Fluosol DA is heated in a bain-marie to a temperature between 20° and 120°. The rise in temperature has the aim of increasing the vapour pressure of the Fluosol DA and thus the amount of oxygen carrier gas in the circuit which is directed towards the skin.

Oxygen and/or nitrogen monoxide, commercially available in the form of cylinders, can also be mixed with the Fluosol DA vapour by injection into the gas mixing chamber (24).

Operation of the pump (9) will aspirate the fluorocarbon/oxygen and/or nitrogen monoxide gas mixture at atmospheric pressure, and direct it at hyperbaric pressure towards the skin via the spray nozzle (28).

The spraying pressure is set to its highest level so as to ensure the greatest penetration of gas molecules while remaining below the pain threshold of the subject. In one particular example, the pressures used are between 1 and 5 bar.

In one particular example, the flow rates used are between 1 to 300 litres per minute and more often between 5 and 50 litres per minute. The duration of this step is around ten minutes.

Other gases can be used, such as ozone or chlorine for their disinfecting properties. In the same way, any chemical molecule which is able to have a sufficient vapour pressure can be used in a precise therapeutic context. This is the case for phenol, camphor, menthol, essential oils, etc.

1. Method for medical, physiotherapeutic or cosmetic treatment of an area of the skin and subjacent organs of a human or animal subject, comprising the step of spraying a pressurised gaseous mixture onto the area, by means of a spray nozzle (28).

2. Method according to claim 1, wherein the gaseous mixture comprises fluorocarbons.

3. Method according to claim 2, wherein the gaseous mixture comprises at least one additional gas having a therapeutic property.

4. Method according to claim 1, wherein the pressure is between 0.01 and 20 bar, more often between 1 and 10 bar and most often between 3 and 5 bar.

5. Method according to claim 1, further comprising the step consisting in partially aspirating excess sprayed gas with a view to recovering it and recycling it.

6. Method according to claim 1, wherein the step of in spraying a pressurised gas onto the area, by means of a spray nozzle (28), is preceded by a step of peeling the superficial epidermis.

7. Device for percutaneous treatment of an area of skin, comprising a handpiece (8) and a spray nozzle (28) mounted on said handpiece.

8. Device according to claim 7, comprising pressurisation means, said pressursation means comprising a suction and compression pump (9).

9. Device according to claim 8, wherein the pressurisation means comprises a flask (3) which is able to receive fluorocarbons and heat them in a bain-marie (26).

10. Device according to claim 8, wherein it comprises a mixing chamber (24) for mixing gases.

11. Device according to claim 7, further comprising filtration means.

12. Handpiece for a device according to claim 7, comprising control means designed to stop pressurised gas by means of a valve (30) in a rest position of said handpiece, an adjustment knob (31) for adjusting a thermostat (16) which controls the temperature of sprayed gas, and a control knob (32) for controlling an electrovalve (14) which regulates flow rate of the sprayed gas.
